(19) United States
(12) Patent Application Publication
(10) Pub. No.: US 2008/0154238 A1
(43) Pub. Date: Jun. 26, 2008

(54) Endometrial ablation device

(76) Inventor: James F. McGuckin, Radnor, PA (US)

Correspondence Address: Neil D. Gershon, 1011 High Ridge Road, Stamford, CT, 06905, US

(21) Appl. No.: 11/998,384

(22) Filed: Nov. 29, 2007

Related U.S. Application Data
(60) Provisional application No. 60/872,520, filed on Dec. 1, 2006.

(51) Int. Cl. A61M 31/00

(52) U.S. Cl. 604/515; 604/103.01

(57) ABSTRACT

An endometrial ablation device comprising a shaft and inner and outer balloons connected to the shaft. The outer balloon has a plurality of pores on an outer surface. The inner balloon and outer balloon are independently inflatable such that the inner balloon is initially inflated to substantially fill the uterine cavity and the outer balloon is inflated subsequently. The outer balloon is filled with a treatment fluid which exits through the pores of the outer balloon.

This application claims priority from provisional application Ser. No. 60/872,520 filed Dec. 1, 2006, the entire contents of which is incorporated herein by reference.

### BACKGROUND

1. Field of the Invention

This application relates to an endometrial ablation device, and more particularly to an endometrial ablation device for delivering ablation fluid to the uterine wall.

2. Background of Related Art

Endometrial ablation involves ablation of the endometrium to destroy one or more of the layers on the inner lining. Such destruction is used as an alternative to major surgery in the treatment or prevention of certain diseases. Dysfunctional uterine bleeding is one condition in which endometrial ablation is used.

Various techniques are utilized to apply thermal energy to the lining. These include utilizing an electrode or laser inserted into the endometrial cavity through a scope to deliver energy to the walls. In some devices, RF or microwave balloon electrodes are utilized for thermal ablation.

The need exists for an improved endometrial ablation device.

### SUMMARY

The present invention provides an endometrial ablation device comprising a shaft, an inner balloon connected to the shaft and an outer balloon connected to the shaft. The outer balloon has a plurality of pores on an external surface. The inner balloon and outer balloon are independently inflatable such that the inner balloon is initially inflated to substantially fill the uterine cavity and the outer balloon is subsequently inflated. The outer balloon is filled with a treatment fluid and the treatment fluid exits through the pores of the outer balloon.

In one embodiment, an anchor locks the shaft in position. The anchor can be slidable on an external surface of the shaft.

The outer balloon preferably applies pressure against the uterine wall to induce the treatment agent into the wall.

In one embodiment, the shaft has a first hole communicating with the inner balloon and a second hole communicating with the outer balloon.

The present invention also provides an endometrial ablation device comprising an inner balloon and an outer balloon, the inner balloon being inflatable to substantially fill the uterine cavity. The outer balloon is inflatable to substantially conform to the shape of an internal wall of the uterine cavity, wherein the outer balloon is inflated at a pressure to apply pressure against the uterine wall, the pressure causing a treatment agent to pass from an inside of the outer balloon through a series of pores in the outer surface of the balloon and into the uterine wall to cause ablation.

The present invention also provides a method of ablating the lining of the uterus to treat prolonged bleeding. The method comprises the steps of providing a device having an inner and outer balloon, the outer balloon having a plurality of pores, inserting the device through the cervix into the uterine cavity, expanding the inner balloon to distend the uterine cavity and anchor the device, and subsequently expanding the outer balloon with a treatment fluid so that the wall of the outer balloon applies a pressure against a wall of the uterine cavity to cause the treatment fluid to flow through the pores and into the uterine wall.

The method may further comprise the step of moving a locking mechanism to secure the device.

The method may further comprise the step of occluding the fallopian tube prior to the step of expanding the outer balloon.

### BRIEF DESCRIPTION OF THE DRAWINGS

Preferred embodiment(s) of the present disclosure are described herein with reference to the drawings wherein:

FIG. 1 is an anatomical drawing of the uterus and fallopian tubes;

FIG. 2 illustrates a guide wire inserted into the uterine cavity;

FIG. 3 illustrates insertion of the double balloon endometrial ablation device of the present invention over the guidewire;

FIG. 4 illustrates the inner balloon of the ablation device inflated;

FIG. 5 is a close up view of the device of FIG. 4;

FIG. 6 illustrates the porous outer balloon filled with ablation fluid;

FIG. 7 illustrates penetration of ablation fluid from the outer balloon into the uterine wall;

FIG. 8 is a close up view illustrating penetration of the ablation fluid from the outer balloon into the uterine wall by the pressure gradient; and

FIG. 9 is a view similar to FIG. 6 except showing occlusion elements in the fallopian tube.

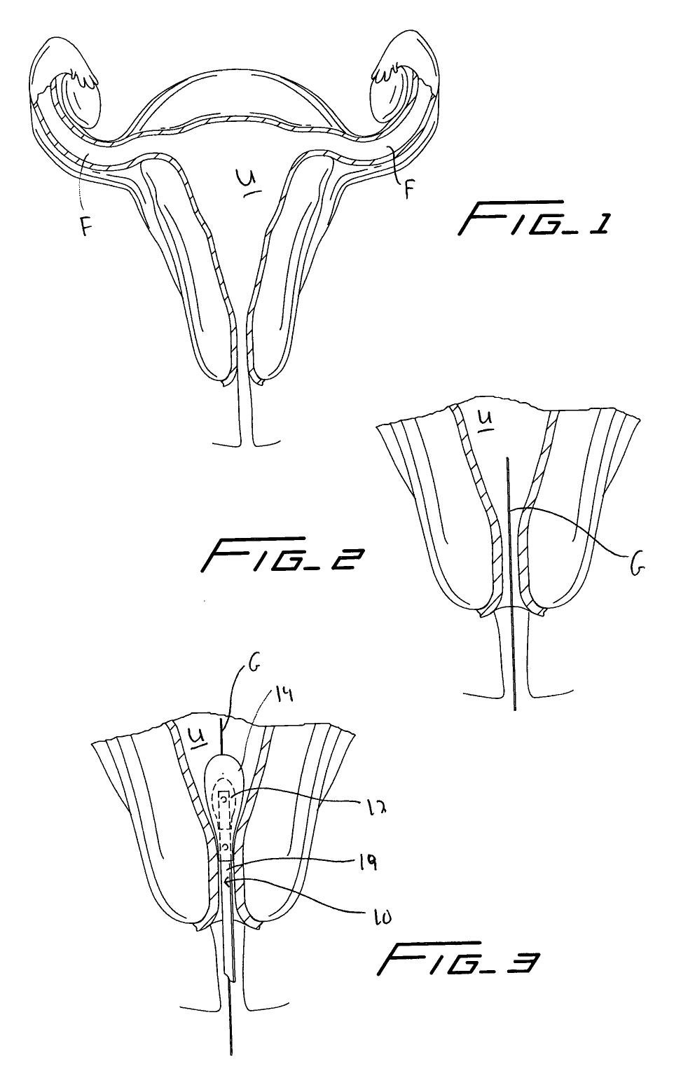

### DETAILED DESCRIPTION OF PREFERRED EMBODIMENTS

Referring now in detail to the drawings where like reference numerals identify similar or like components throughout the several views, the endometrial ablation device of the present invention is illustrated. The device is designed to ablate the lining of the uterus to cease menses for prolonged bleeding. The device provides a wall contact method to not only deliver an agent to the uterine wall in a more controlled manner but allow for the ablative agent to penetrate the wall. Agents that can be applied include ethanol, acetic acid or other chemical or thermally caustic agents.

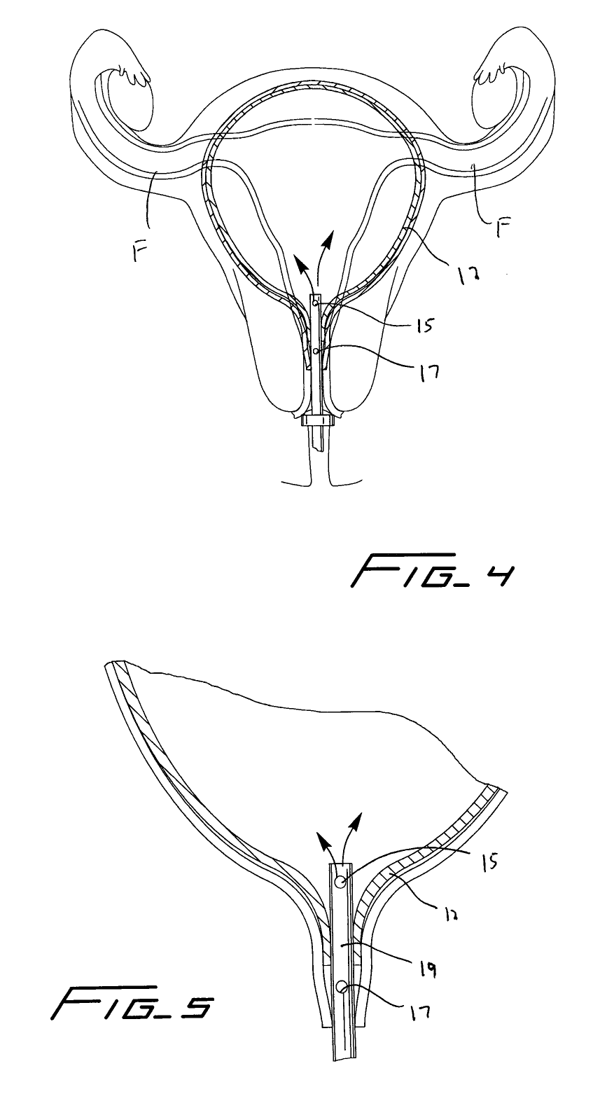

The device 10, shown in FIGS. 2-7, comprises an inner balloon 12 and a compliant and porous outer balloon 14 designed to leak the ablative agent. The balloons 12 and 14 are inflated independently. An elongated rod or shaft 19 supports the balloons 12 and 14 and contains openings for passage of fluid to inflate the balloons. The device is inserted over a guidewire G, shown in FIG. 2, extending through the cervix and into the uterine cavity U. Preferably, the guidewire G is inserted by direct visualization. As shown in FIG. 3, the device 10 is inserted over the guidewire G into the uterine cavity with both inner balloon 12 and outer balloon 14 in the uninflated/unfilled collapsed position.

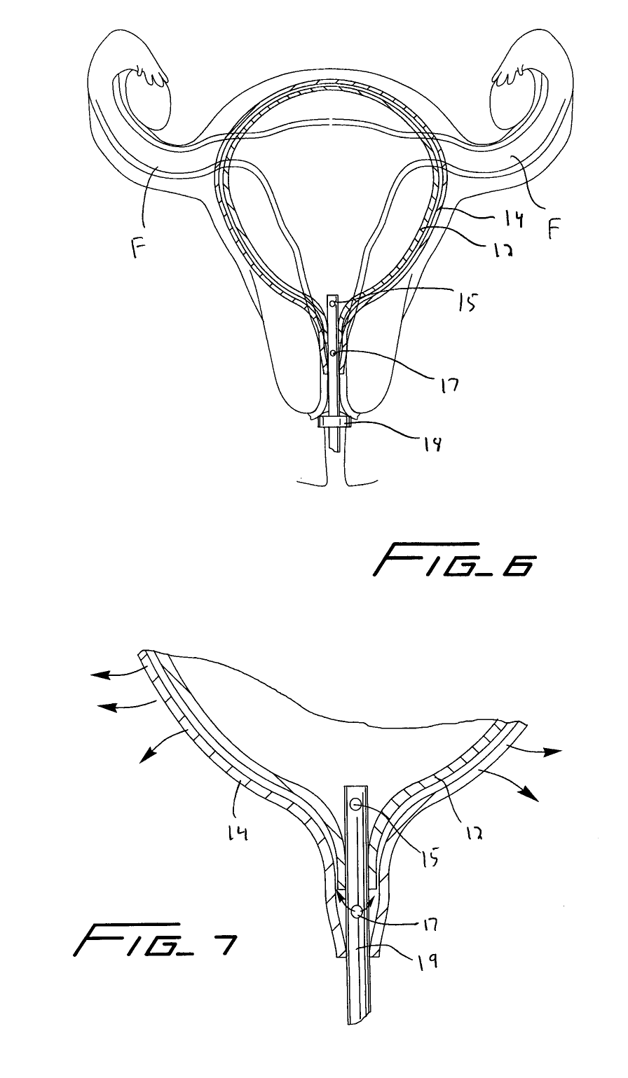

FIGS. 4 and 5 illustrate the inner balloon 12 inflated by fluid exiting through holes 15 in shaft 14, represented by the arrows, to decrease the space in the intrauterine cavity U, distend the cavity and act as an internal lock or anchor on the cervix to prevent leakage of fluids into the vagina. It also acts as a tethering mechanism. That is, the uterus is typically distorted or irregular shaped so by filling the balloon it occupies most of the space in the uterus. As shown, holes 17 in shaft 14 are not in communication with the inner balloon 12, but rather communicate with outer balloon 14 as described below.

After the inner balloon 12 is inflated, it is pulled back against the cervix and a cervical anchor 18, shown in the form of a disk or collar, is advanced and locked to secure device 10 in place as shown in FIG. 6. The anchor 18 is preferably positioned around the outer surface of the shaft 14 for movement thereover. The outer balloon 14 remains in the unfilled condition.

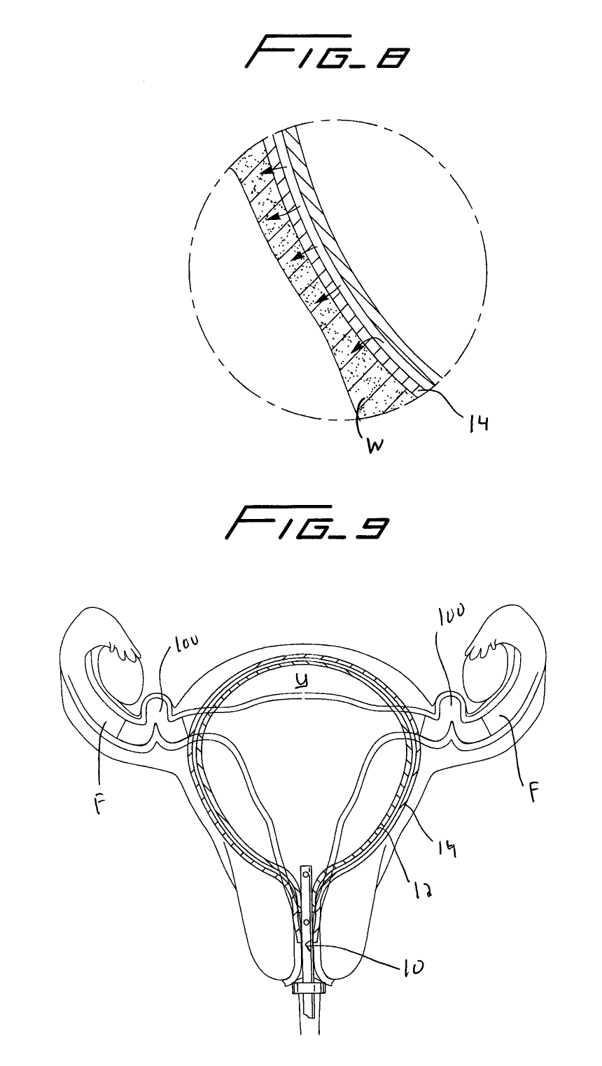

Next, the outer balloon 14 is filled with ablation fluid, such as acetic acid or ethanol. The fluid exits though holes 17 in shaft 19 as shown in FIG. 7. The outer balloon 14 is compliant and directly contacts the uterine wall as it fills the space between the inner balloon 12 and the uterine wall, preferably conforming to the shape of the internal wall of the cavity. The pores on the balloon 14 allow the acetic acid to exit from inside the balloon and out through the outer surface of the balloon. Note that the fallopian tubes F would be occluded to prevent passage of the ablative agent. One device for occluding the fallopian tubes is disclosed in commonly assigned co-pending patent application Ser. No. 60/872,382 entitled Fallopian Tube Occlusion Device, filed on Dec. 1, 2006, and co-pending application entitled Fallopian Tube Occlusion Device, filed the same day as the filing of this application. The entire contents of these applications are incorporated herein by reference. This fallopian tube occlusion device is shown in FIG. 9 and designated by reference numeral 100. Other methods and devices for occluding the fallopian tubes could also be utilized.

The inner and/or outer balloons provide pressure against the endometrial cavity to induce the chemical or thermal agent into the uterine wall W (see FIG. 8). That is, the agent is driven by the pressure gradient into the uterine wall W causing ablation of endometrial tissue and necrosis of the layers. Scarring causes cessation of menses.

After the procedure, the ablation fluid is aspirated from the outer balloon 14, causing it to collapse, followed by deflation of the inner balloon 12, to enable withdrawal of the device 10 from the uterus.

While the above description contains many specifics, those specifics should not be construed as limitations on the scope of the disclosure, but merely as exemplifications of preferred embodiments thereof. Those skilled in the art will envision many other possible variations that are within the scope and spirit of the disclosure.

What is claimed is:

1. An endometrial ablation device comprising a shaft, an inner balloon connected to the shaft and an outer balloon connected to the shaft, the outer balloon having a plurality of pores on an outer surface, wherein the inner balloon and outer balloon are independently inflatable such that the inner balloon is initially inflated to substantially fill the uterine cavity and the outer balloon is inflated subsequently, the outer balloon filled with a treatment fluid, the treatment fluid exiting through the pores of the outer balloon.

2. The device of claim 1, further comprising an anchor to lock the shaft in position.

3. The device of claim 2, wherein the anchor is slidable on an external surface of the shaft.

4. The device of claim 3, wherein the anchor is in the form of a disk.

5. The device of claim 1, wherein the outer balloon applies pressure against the uterine wall to induce the treatment agent into the wall.

6. The device of claim 1, wherein the shaft has a first hole communicating with the inner balloon and a second hole communicating with the outer balloon.

7. The device of claim 1, wherein the outer balloon is composed of a compliant material.

8. An endometrial ablation device comprising an inner balloon and an outer balloon, the inner balloon being inflatable to substantially fill the uterine cavity, the outer balloon being inflatable to substantially conform to the shape of an internal wall of the uterine cavity, wherein the outer balloon is inflated at a pressure to apply pressure against the uterine wall, the pressure causing a treatment agent to pass from an inside of the outer balloon through a series of pores in the outer surface of the balloon and into the uterine wall to cause ablation.

9. The device of claim 8, wherein the shaft has a first hole communicating with the inner balloon and a second hole communicating with the outer balloon.

10. The device of claim 8, further comprising an anchor to lock the shaft in position, wherein the anchor is slidable on an external surface of the shaft.

11. A method of ablating the lining of the uterus to treat prolonged bleeding, the method comprising the steps of:
a) providing a device having an inner and outer balloon, the outer balloon having a plurality of pores;
b) inserting the device through the cervix into the uterine cavity;
c) expanding the inner balloon to distend the uterine cavity and anchor the device; and
d) subsequently expanding the outer balloon with a treatment fluid so that the wall of the outer balloon applies a pressure against a wall of the uterine cavity to cause the treatment fluid to flow through the pores and into the uterine wall.

12. The method of claim 11, further comprising the step of moving a locking mechanism to secure the device.

13. The method of claim 11, further comprising the step of occluding the fallopian tube prior to the step of expanding the outer balloon.
